(19) United States
(12) Patent Application Publication
(10) Pub. No.: US 2007/0025877 A1
(43) Pub. Date: Feb. 1, 2007

(54) User interface for portable medical diagnostic apparatus and method of using the same

(76) Inventor: Christian M. Hansen, Medway, MA (US)

Correspondence Address: Toby H Kusmer;McDermott Will & Emery, 28 State Street, Boston, MA, 02109-1775, US

(21) Appl. No.: 10/558,815

(22) PCT Filed: Jun. 3, 2004
(86) PCT No.: PCT/US04/17345

§ 371 Date: Nov. 29, 2005

Related U.S. Application Data
(60) Provisional application No. 60/475,352, filed on Jun. 3, 2003.

(51) Int. Cl. G01N 33/00

(52) U.S. Cl. 422/68.1; 422/58

(57) ABSTRACT

A medical diagnostic apparatus including a display screen for displaying a graphical user interface (GUI) including virtual switches, a user input device for allowing the virtual switches of the GUI to be selected, a processor connected to the display screen and the user input device, and a computer program having alphanumeric input instructions that cause the processor to display one of a numeric entry GUI and an alphanumeric entry GUI on the display screen. Selecting a predetermined virtual switch of the numeric GUI using the input device causes the alphanumeric GUI to be displayed, while selecting a predetermined virtual switch of the alphanumeric GUI causes the numeric GUI to be displayed.

### CROSS-REFERENCE TO RELATED APPLICATIONS

The present application claims priority from co-pending provisional U.S. Patent Application Ser. No. 60/475,352, filed Jun. 3, 2003 (Attorney docket number BYRK-28PR), which is incorporated herein by reference in its entirety.

### FIELD OF THE DISCLOSURE

The present disclosure relates to a medical diagnostic instrument and, more particularly, to a glucose meter, a user interface for a glucose meter and a method of operating a glucose meter. Even more particularly, the present disclosure relates to a portable glucose meter having a touch screen liquid crystal display and a user interface permitting the entry of alphanumeric data through the touch screen.

### BACKGROUND OF THE DISCLOSURE

Blood glucose meters are medical diagnostic instruments used to measure the level of glucose in a patient's blood. Some meters determine glucose levels by measuring the amount of electricity that can pass through a sample of blood, and other meters measure how much light reflects from the sample. The meter then uses the amount of light or electricity to compute the glucose level and displays the glucose level as a number. Generally, to operate a blood glucose meter, a patient or caregiver, such as a nurse or doctor, deposits a drop of the patient's blood onto a disposable cartridge or pad. The disposable cartridge along with the drop of blood is then inserted into a slot located on the blood glucose meter, whereupon the blood glucose meter tests the blood located on the disposable cartridge in order to determine the level of glucose in the blood. Upon determining the level of glucose in the blood, the blood glucose meter displays this information along with other information on a screen located on the blood glucose meter. Many glucose meters also include switches for allowing a user to input information or queries into the meter. Preferably, glucose meters are small enough and light weight enough to be portable and conveniently carried by a user.

Since it is important that a glucose meter is small and light weight enough to be easily carried (e.g., about the size of a personal digital assistant or a cellular telephone), any user interface components of glucose meter should also be relatively small. For example, if a glucose meter includes a screen, such as a liquid crystal display (LCD), for displaying information to a user, the screen should be a small and light weight as possible. In addition, if a glucose meter includes switches for allowing a user to input information or queries into the meter, the switches should each be as small as possible and there should be as few switches as possible (e.g., a portable glucose meter is generally too small to house a full alphanumeric keyboard.

In addition to being portable, it is desirable that a glucose meter is relatively inexpensive and easy to use. For example, if a glucose meter is relatively expensive, healthcare providers and medical insurers may limit the patient populations approved to use the glucose meter and therapies for which the glucose meter can be used. In addition, if a glucose meter is relatively difficult to operate, many patients who qualify as potential users may be resistant to using the glucose meter.

What is still desired, therefore, is a new and improved medical diagnostic apparatus, such as a glucose meter. Preferably, the new and improved glucose meter will be small enough and light weight enough to be portable and conveniently carried by a user. In addition, the new and improved glucose meter will preferably include user interface components for displaying information and queries to a user and for receiving information and queries from a user. Preferably, the glucose meter will have a new and improved user interface that will allow a user to easily and intuitively program, operate and obtain feedback from the glucose meter.

### SUMMARY OF THE DISCLOSURE

The present disclosure is directed to exemplary embodiments of a new and improved medical diagnostic apparatus, a user interface for a medical diagnostic apparatus and a method of operating a medical diagnostic apparatus.

One exemplary embodiment of the medical diagnostic apparatus includes a display screen for displaying a graphical user interface (GUI) including virtual switches, a user input device for allowing the virtual switches of the GUI to be selected, a processor connected to the display screen and the user input device, and a computer program having alphanumeric input instructions that cause the processor to display one of a numeric entry GUI and an alphanumeric entry GUI on the display screen, wherein selecting a predetermined virtual switch of the numeric GUI using-the input device causes the alphanumeric GUI to be displayed, while selecting a predetermined virtual switch of the alphanumeric GUI causes the numeric GUI to be displayed.

Another exemplary embodiment of the medical diagnostic apparatus includes a display screen for displaying a GUI including virtual switches, a user input device for allowing the virtual switches of the GUI to be selected, a processor connected to the display screen and the user input device, and a computer program having alphanumeric input instructions that cause the processor to display an alphanumeric entry GUI on the display screen. The alphanumeric GUI includes ten virtual switches labeled with numeric characters 0-9 respectively, and futrther includes multiple sets of virtual switches having virtual switches labeled with alphabetical characters. Each set of virtual switches labeled with alphabetical characters is associated with one of the ten virtual switches labeled with numeric characters, and the computer program is programmed to display one of the sets when the numeric character virtual switch associated with the set is selected.

Among other aspects, benefits and advantages of the present disclosure, a GUI according to the present disclosure allows both numerical and alphabetical characters to be entered into a medical diagnostic apparatus, such as a blood glucose meter, without requiring a large display screen and keyboard. A GUI according to the present disclosure, therefore, allows a medical diagnostic apparatus to remain small enough and light weight enough to be portable and conveniently carried by a user, yet allows the user interface of the apparatus to be relatively sophisticated. A GUI according to the present disclosure also allows a user to easily and intuitively program, operate and obtain feedback from the medical diagnostic apparatus.

Additional aspects, benefits and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

Reference is made to the attached drawings, wherein elements having the same reference character designations represent like elements throughout, and wherein:

FIG. 1 is a perspective top end view of an exemplary embodiment of a handheld glucose meter having a touch screen in combination with a liquid crystal display (LCD);

FIG. 2 is a perspective top end view, in section, of the handheld glucose meter of FIG. 1;

FIG. 3 is a perspective front view of the handheld glucose meter of FIGS. 1 and 2;

FIG. 4A is a screen shot showing exemplary embodiments of an alphanumeric entry graphical user interface (GUI) according to the present disclosure for display on the LCD of the handheld glucose meter of FIGS. 1-3;

FIG. 4B are screen shots showing exemplary embodiments of a numeric entry GUI and an alphanumeric entry GUI according to the present disclosure for display on the LCD of the handheld glucose meter of FIGS. 1-3 wherein, as illustrated, pressing the touch screen above a virtual switch labeled “ABC . . . ” of the numeric GUI causes the alphanumeric GUI to be displayed, while pressing the touch screen above a virtual switch labeled “123 . . . ” of the alphanumeric GUI causes the numeric GUI to be displayed;

FIG. 5 is a series of screen shots showing exemplary embodiments of a “home menu” and a “patient test” GUI according to the present disclosure for display on the LCD of the handheld glucose meter of FIGS. 1-3, wherein the patient test GUI is selected and run from the home menu GUI, and wherein the patient test offers a selection between the numeric GUI and the alphanumeric GUI of FIG. 4B;

FIG. 6 is a series of screen shots showing an exemplary embodiment of a “quality control test” GUI according to the present disclosure for display on the LCD of the handheld glucose meter of FIGS. 1-3, wherein the quality control test GUI is selected and run from the home menu GUI, and wherein the quality control test offers a selection between the numeric GUI and the alphanumeric GUI of FIG. 4B;

FIG. 7 is a series of screen shots showing exemplary embodiment of a “meter” and a “linearity test” GUI according to the present disclosure for display on the LCD of the handheld glucose meter of FIGS. 1-3, wherein the linearity test GUI is selected and run from the meter GUI, and the meter GUI is selected and run from the home menu GUI, and wherein the linearity test offers a selection between the numeric GUI and the alphanumeric GUI of FIG. 4B; and

FIG. 8 is a series of screen shots showing an exemplary embodiment of a “proficiency test” GUI according to the present disclosure for display on the LCD of the handheld glucose meter of FIGS. 1-3, wherein the proficiency test GUI is selected and run from the meter GUI, and wherein the proficiency test offers a selection between the numeric GUI and the alphanumeric GUI of FIG. 4B.

### DETAILED DESCRIPTION OF EXEMPLARY EMBODIMENTS

The present disclosure is directed to exemplary embodiments of a new and improved medical diagnostic apparatus, a user interface for a medical diagnostic apparatus and a method of operating a medical diagnostic apparatus. Among other aspects, benefits and advantages of the present disclosure, a user interface according to the present disclosure allows both numerical and alphabetical characters to be entered into a medical diagnostic apparatus, such as a blood glucose meter, without requiring a large display screen or keyboard. A user inter-face according to the present disclosure, therefore, allows a medical diagnostic apparatus to remain small enough and light weight enough to be portable and conveniently carried by a user, yet allows the user interface of the apparatus to be relatively sophisticated. A user interface according to the present disclosure also allows a user to easily and intuitively program, operate and obtain feedback from the medical diagnostic apparatus.

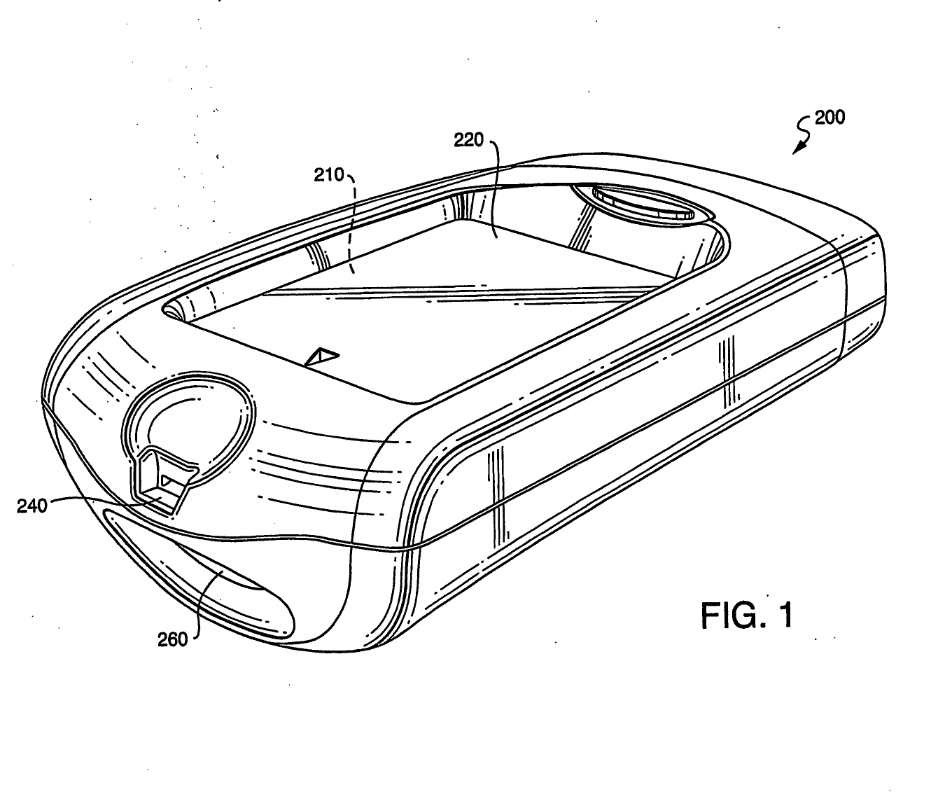

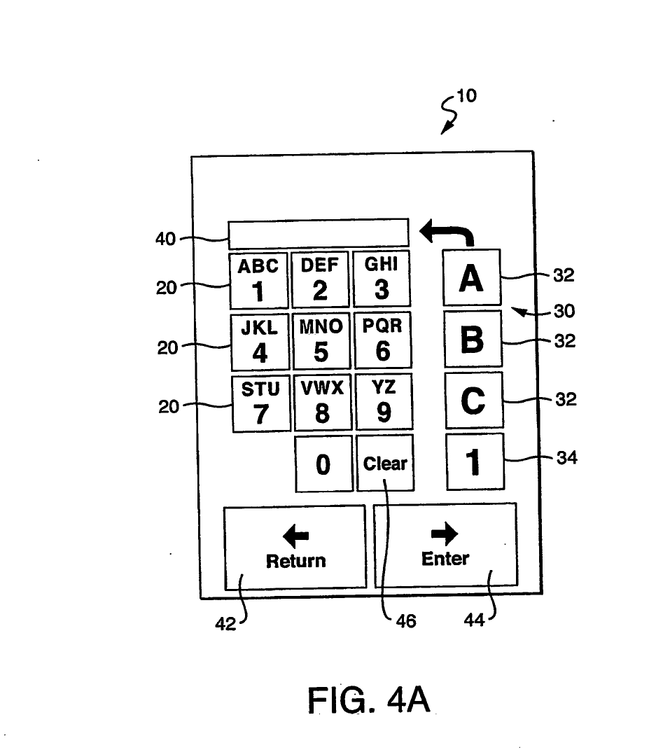

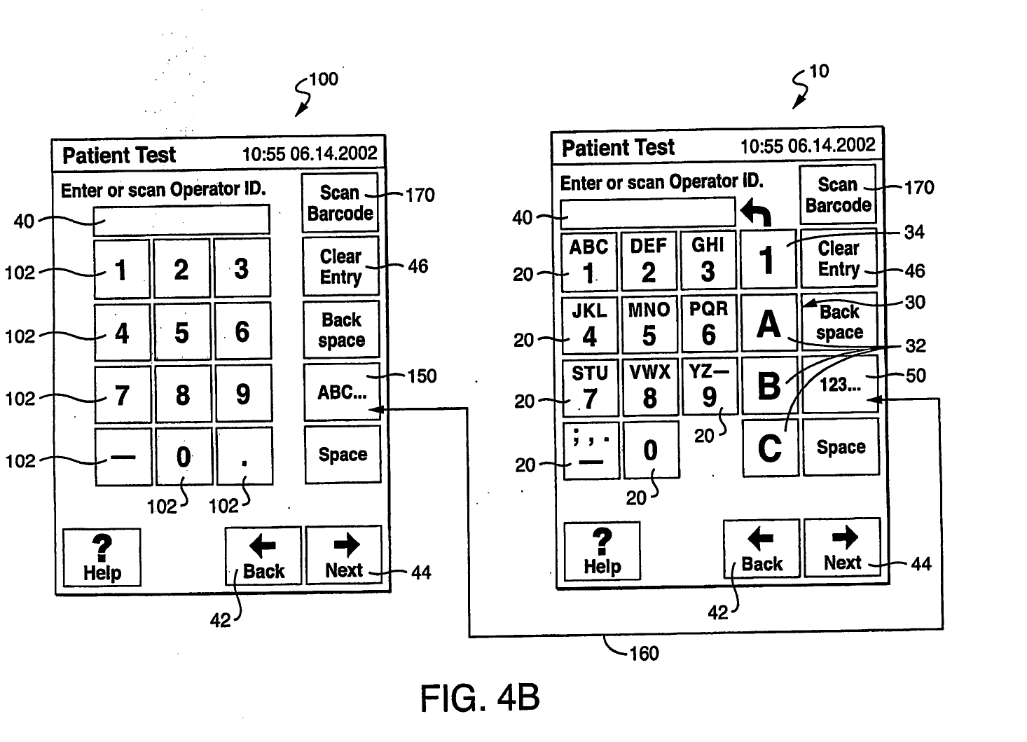

Referring to FIG. 4A, one exemplary embodiment of an alphanumeric entry graphical user interface (GUI) 10 according to the present disclosure is adapted for display on a display screen of a medical diagnostic apparatus and includes virtual switches that can be selected using a user input device of the medical diagnostic apparatus. The medical diagnostic apparatus will also include a processor connected to the display screen and the user input device, and a computer program having alphanumeric input instructions that cause the processor to display the alphanumeric entry GUI on the display screen. FIGS. 1-3 show an exemplary embodiment of a handheld medical diagnostic apparatus 200 that can utilize the alphanumeric entry GUI 10 of FIG. 4A, and will be discussed in detail below. However, it should be understood that an alphanumeric entry GUI according to the present disclosure can be used with a medical diagnostic apparatus other than the particular apparatus 200 shown in FIGS. 1-3.

The alphanumeric GUI 10 of FIG. 4A includes ten virtual switches 20 labeled with numeric characters 0-9 respectively, and further includes multiple sets 30 of virtual switches that are alternately displayed and have virtual switches 32 labeled with alphabetical characters. Each set 30 of virtual switches 32 labeled with alphabetical characters is associated with one of the ten virtual switches 20 labeled with numeric characters, and the computer program is programmed to instruct the processor to display only one of the sets 30 when the numeric character virtual switch 20 associated with the set 30 is selected.

In the exemplary embodiment of FIG. 4A, the sets 30 of virtual switches of the alphanumeric entry GUI include:
1.) a set of virtual switches 32 labeled with A, B, C, respectively, associated with the virtual switch 20 labeled with numeric character 1 (shown in FIG. 4A);
2.) a set of virtual switches labeled with D, E, F, respectively, associated with the virtual switch 20 labeled with numeric character 2;
3.) a set of virtual switches labeled with G, H, I, respectively, associated with the virtual switch 20 labeled with numeric character 3;
4.) a set of virtual switches labeled with J, K, L, respectively, associated with the virtual switch 20 labeled with numeric character 4;
5.) a set of virtual switches labeled with M, N, O, respectively, associated with the virtual switch 20 labeled with numeric character 5;
6.) a set of virtual switches labeled with P, Q, R, respectively, associated with the virtual switch 20 labeled with numeric character 6;
7.) a set of virtual switches labeled with S, T, U, respectively, associated with the virtual switch 20 labeled with numeric character 7;
8.) a set of virtual switches labeled with V, W, X, respectively, associated with the virtual switch 20 labeled with numeric character 8; and
9.) a set of virtual switches labeled with Y, Z, respectively, associated with the virtual switch labeled 20 with numeric character 9.

In the exemplary embodiment of FIG. 4A, each of the sets 30 of virtual switches 32 of the alphanumeric entry GUI also includes a virtual switch 34 labeled with the numeric character associated with the set. For example, and as shown in FIG. 4A, the set 30 of virtual switches 32 labeled with A, B, C, respectively, associated with the virtual switch 20 labeled with numeric character 1 (shown highlighted) also includes a virtual switch 34 labeled with the numeric character 1. In addition, the ten virtual switches 20 labeled with numeric characters 0-9 also are labeled with the alphabetical characters associated with the particular numeric character of the switch. For example, and as shown in FIG. 4A, the virtual switch 20 labeled with numeric character 1 (shown highlighted) also is labeled with the alphabetical characters associated with that switch: A,B and C.

The alphanumeric GUI 10 of FIG. 4A is very small in size. For example, the GUI 10 may occupy a display area of no more than 240 pixels by 320 pixels. The GUI 10 also includes a registry 40, a virtual key 42 labeled “RETURN”, a virtual key 44 labeled “ENTER”, and a virtual key 46 labeled “Clear”.

During operation, a user first selects one of the ten virtual switches 20 labeled with numeric characters 0-9, such that the corresponding set 30 of virtual switches 32, 34 labeled with alphabetical characters and the numeric character are displayed. For example, in FIG. 4A the virtual switch 20 labeled with numeric character 1 has been selected, such that the corresponding set 30 of virtual switches 32,34 labeled with A, B, C and 1 is displayed. A user then can select one of the virtual switches 32, 34 labeled with alphabetical characters and the numeric character (e.g., A, B, C, or 1) and that character will appear in the register 40. If the alphabetical character or numeric character is incorrectly entered in the registry 40, then the user can select the virtual key 46 labeled “Clear” to remove the character from the registry. Otherwise the user may enter another character by selecting one of the virtual switches 32, 34 displayed, or by selecting another of the ten virtual switches 20 labeled with numeric characters 0-9 so that a different set 30 is displayed and selecting one of the virtual switches 32, 34 from the displayed set 30 (for example if the virtual switch 20 labeled with numeric character 5 has been selected, the corresponding set 30 of virtual switches 32, 34 labeled with M, N, O and 5 is displayed). The user may alternatively select the virtual key 44 labeled “ENTER” to save the characters in the registry and move to the next entry page, or select the virtual key 42 labeled “RETURN” to cause a prior entry page to be displayed.

The handheld medical diagnostic apparatus 200 shown in FIGS. 1-3 is one example of a medical diagnostic apparatus that can utilize the alphanumeric entry GUI 10 of FIG. 4A. The handheld medical diagnostic apparatus shown in FIGS. 1-3 actually comprises a glucose meter 200, however, it should be understood that an alphanumeric entry GUI according to the present disclosure can be used with a medical diagnostic apparatus other than a glucose meter.

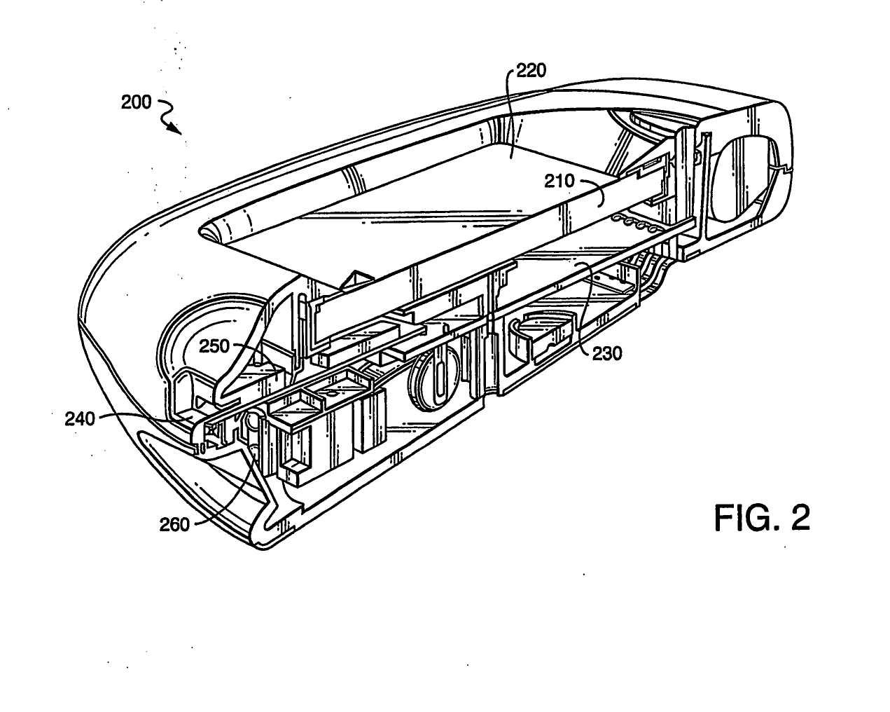

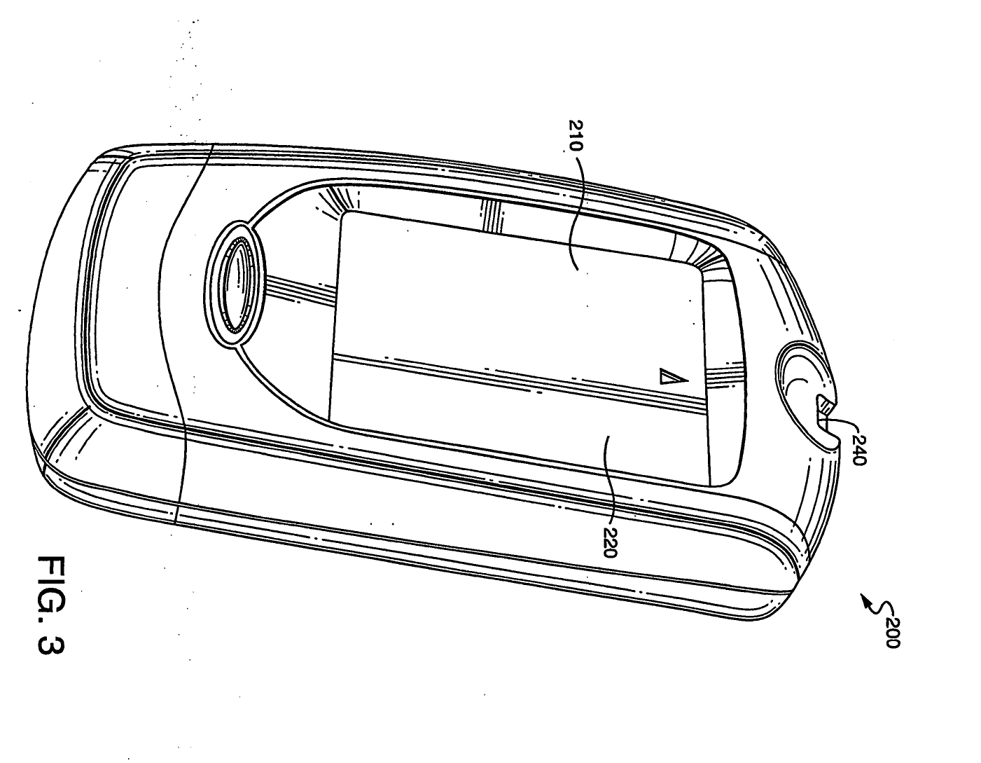

The glucose-meter 200 generally includes a display screen 210, a user input device 220, and a computer processor 230 (shown in FIG. 2). In the exemplary embodiment shown, the display screen comprises a liquid crystal display (LCD) 210 and the user input device comprises a touch screen 220 layered over the LCD. The touch screen 220 allows users to operate the glucose meter 200 by simply touching the screen. Coordinates of the point of contact on the touch screen 220 are calculated by the processor 230 (much as a computer mouse driver translates a mouse's movements into a click or a drag). Other acceptable input devices for use with the GUI of the present disclosure include a trackball, a trackpoint, and a touchpad, all of which have buttons that act like the right and left buttons on a mouse.

In the exemplary embodiment shown in FIGS. 1-3, the glucose meter 200 further includes a port 240 for receiving a fluid sample (i.e., drop of blood placed on disposable cartridge or pad), and a detector 250 (shown in FIG. 2) connected to the processor 230 and adapted to measure a predetermined physical characteristic of the fluid sample received in the port. The detector 250, for example, may measure the amount of electricity that can pass through the sample of blood or may measure how much light reflects from the sample. The processor 230 is programmed to calculate a level of blood glucose in the fluid sample received in the port using the measurement of the physical characteristic of the fluid sample provide by the detector. The glucose meter 200 also includes a bar code scanner 260 connected to the processor, for scanning barcodes off disposable cartridges or pads used with the glucose meter. The glucose meter 200 is small enough and light weight enough to be portable and conveniently carried by a user.

FIG. 4B are screen shots showing exemplary embodiments of a numeric entry GUI 100 according to the present disclosure and an alphanumeric entry GUI 10 according to the present disclosure for alternately being displayed on the LCD 210 of the handheld glucose meter 200 of FIGS. 1-3. The alphanumeric entry GUI 10 of FIG. 4B is similar to the alphanumeric entry GUI 10 of FIG. 4A such that similar elements have the same reference numerals (the GUI 10 of FIG. 4B also includes a virtual switch 20 labeled with “-” and “: , .”). As previously described, the alphanumeric entry GUI 10 allows the entry of both alphabetical and numeric characters from a user.

In contrast, the numeric entry GUI 100 of FIG. 4B allows only the entry of numeric characters from a user. The numeric GUI 100 includes virtual switches 102 labeled with numeric characters 0-9 respectively (the GUI 100 also includes virtual switches 102 labeled with “-” and “.”). As illustrated by arrow 160, selecting a virtual switch 150 labeled “ABC . . . ” of the numeric GUI 100 causes the alphanumeric GUI 10 to be displayed, while selecting a virtual switch 50 labeled “123 . . . ” of the alphanumeric GUI 10 causes the numeric GUI 100 to be displayed. In this manner, a user can select between using the alphanumeric GUI 10 or the numeric GUI 100. As shown, each of the alphanumeric GUI 10 and the numeric GUI 100 includes a virtual switch 170 labeled “Scan Barcode” for activating the barcode scanner 260 of the glucose meter 200 of FIGS. 1-3.

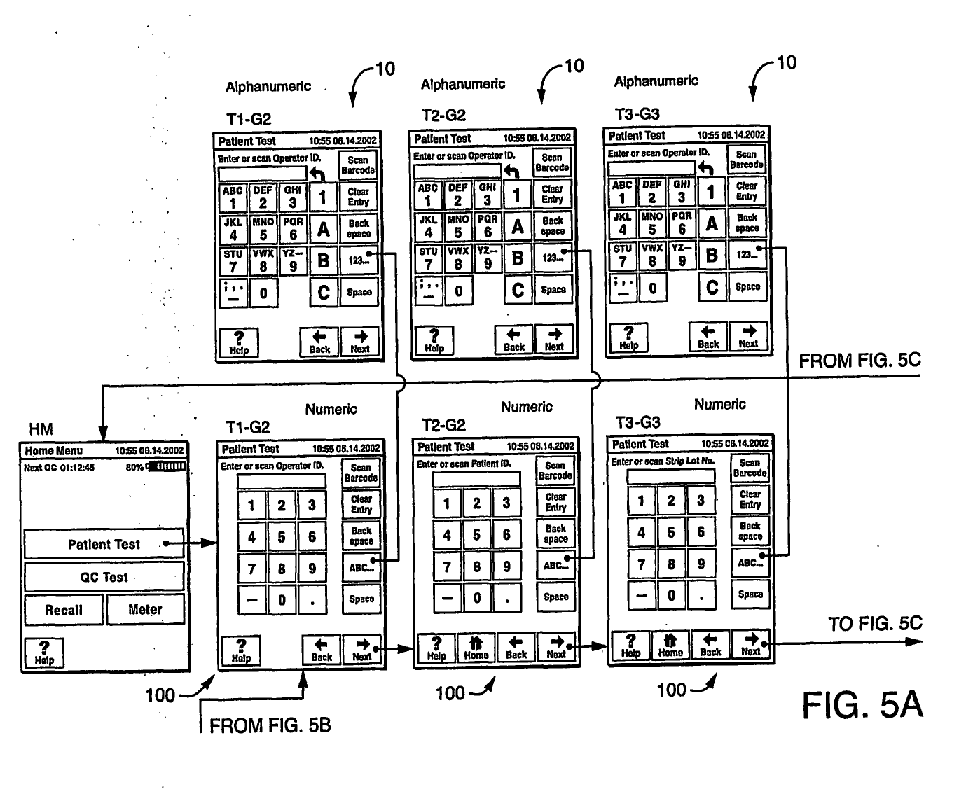

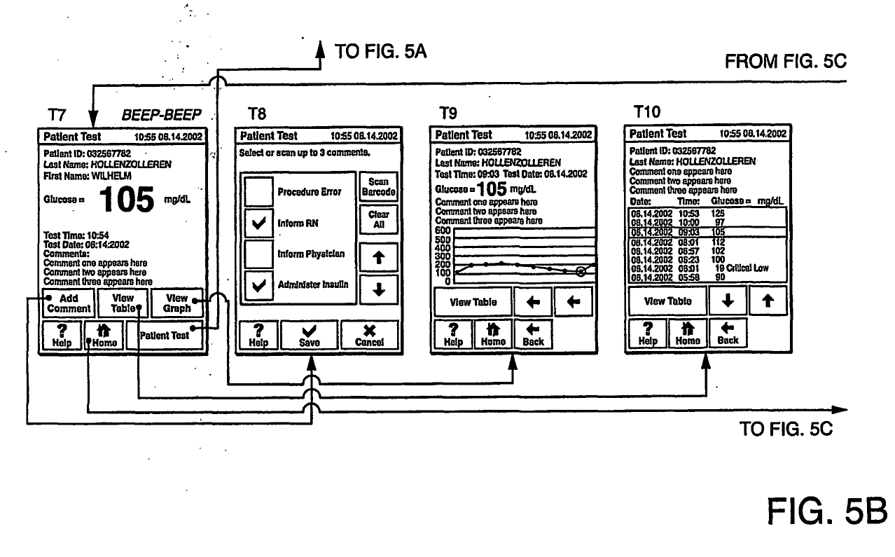

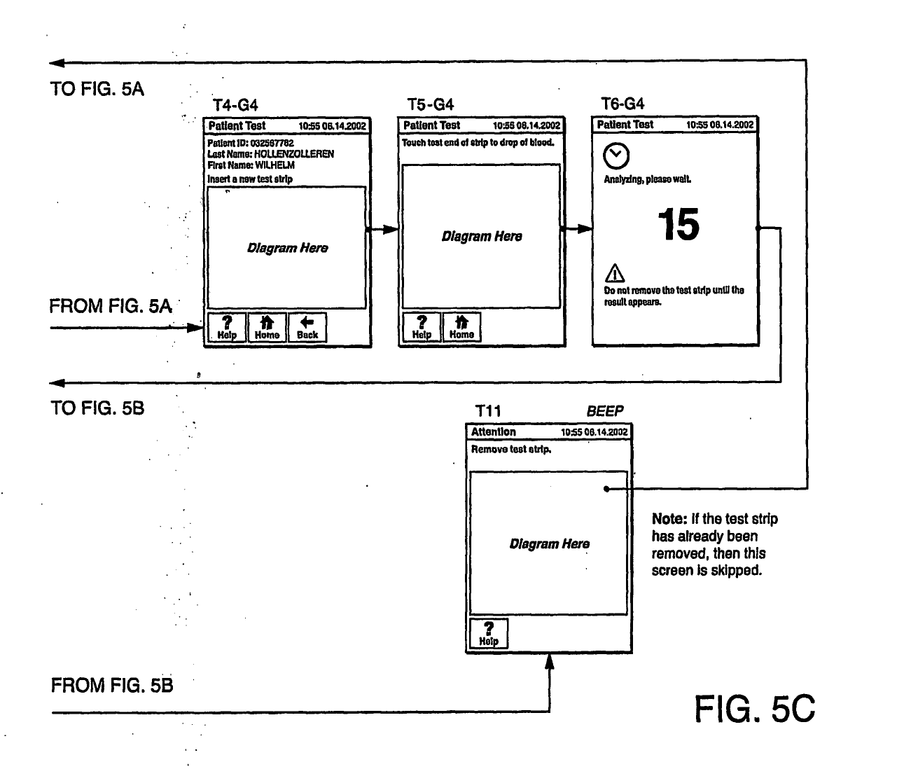

FIG. 5-8 show examples of computer programs used on the handheld glucose meter 200 of FIGS. 1-3 and incorporating the numeric GUI 100 or the alphanumeric GUI 10 of FIG. 4B when data entry is required from the user. FIG. 5, for example, is a series of screen shots showing exemplary embodiments of “home menu” and “patient test” GUIs according to the present disclosure for display on the LCD 210 of the handheld glucose meter 200 of FIGS. 1-3. As shown in FIG. 5, the patient test GUIs are selected and run from the home menu GUI, and the patient test offers a selection between the numeric GUI 100 and the alphanumeric GUI 10 of FIG. 4B when data entry is required from the user (alternatively, just the alphanumeric GUI 10 can be used if desired to receive alphabetical and numeric input).

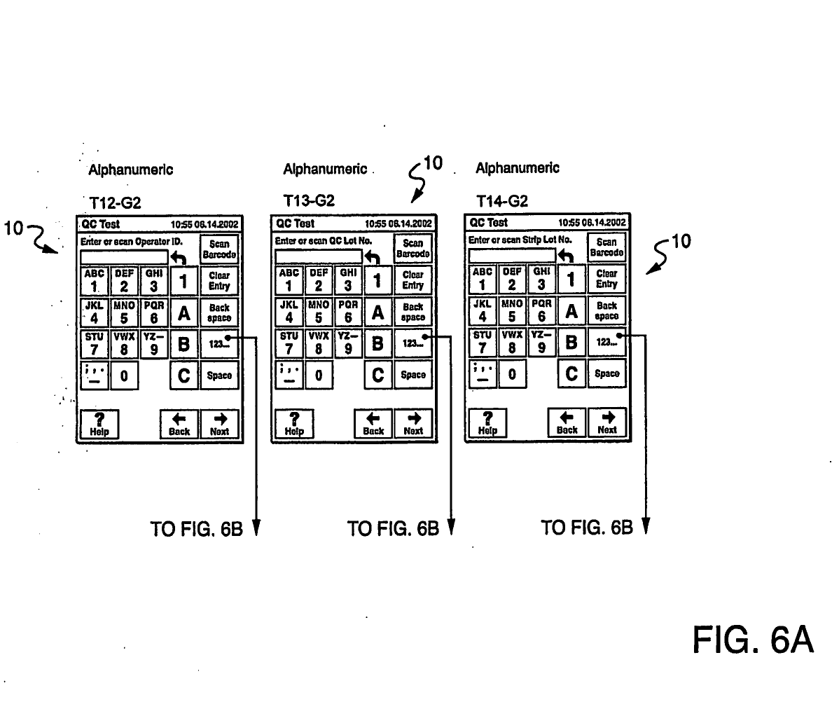

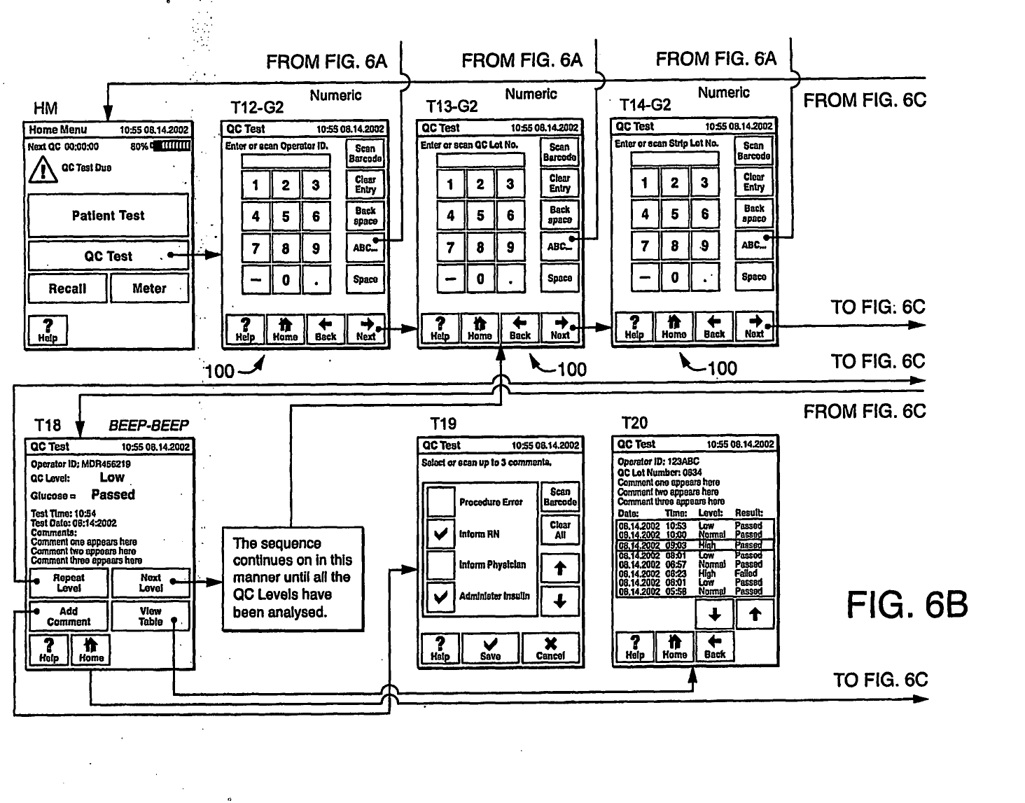

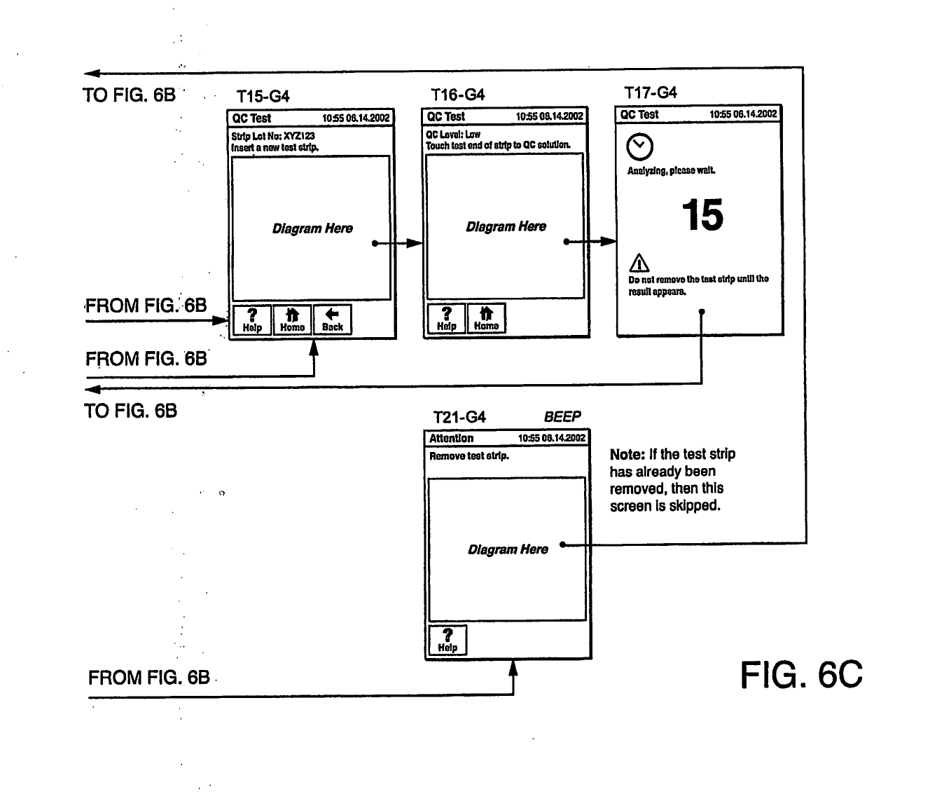

FIG. 6 is a series of screen shots showing an exemplary embodiment of the “home menu” and “quality control test” GUIs according to the present disclosure for display on the LCD 210 of the handheld glucose meter 200 of FIGS. 1-3. As shown in FIG. 6, the quality control test GUIs are selected and run from the home menu GUI, and the quality control test offers a selection between the numeric GUI 100 and the alphanumeric GUI 10 of FIG. 4B when data entry is required from the user (alternatively, just the alphanumeric GUI 10 can be used if desired to receive alphabetical and numeric input).

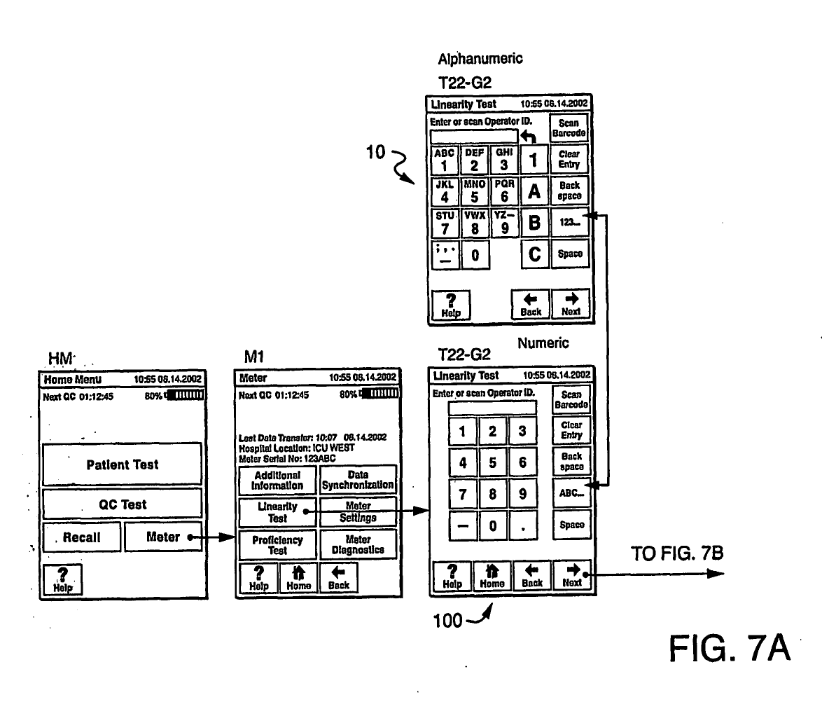

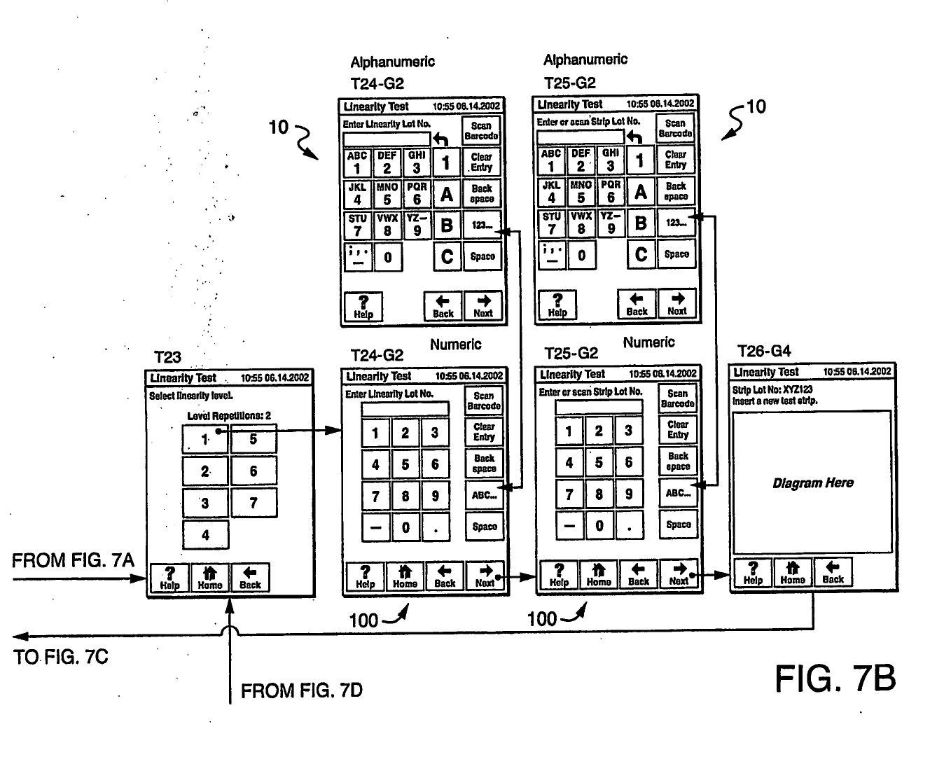

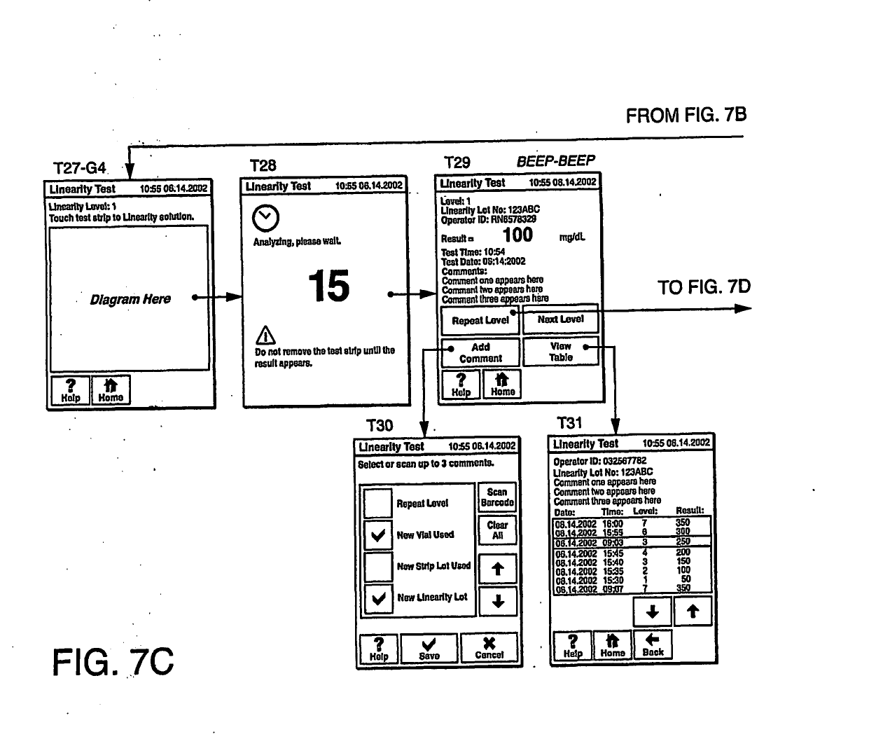

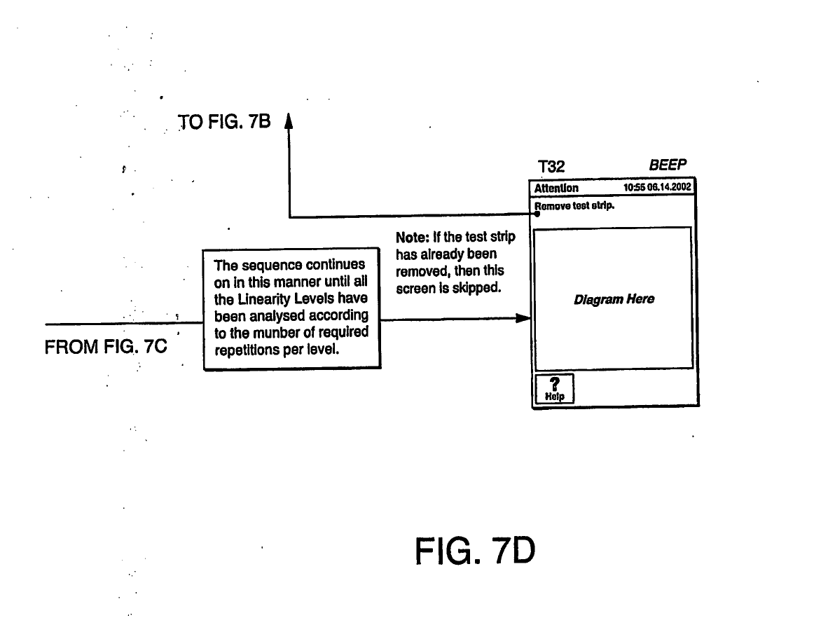

FIG. 7 is a series of screen shots showing exemplary embodiments of “meter” and “linearity test” GUIs according to the present disclosure for display on the LCD 210 of the handheld glucose meter 200 of FIGS. 1-3. As shown in FIG. 7, the linearity test GUIs are selected and run from the meter GUI, and the meter GUI is selected and run from the home menu GUI, and the linearity test offers a selection between the numeric GUI 100 and the alphanumeric GUI 10 of FIG. 4B when data entry is required from the user (alternatively, just the alphanumeric GUI 10 can be used if desired to receive alphabetical and numeric input).

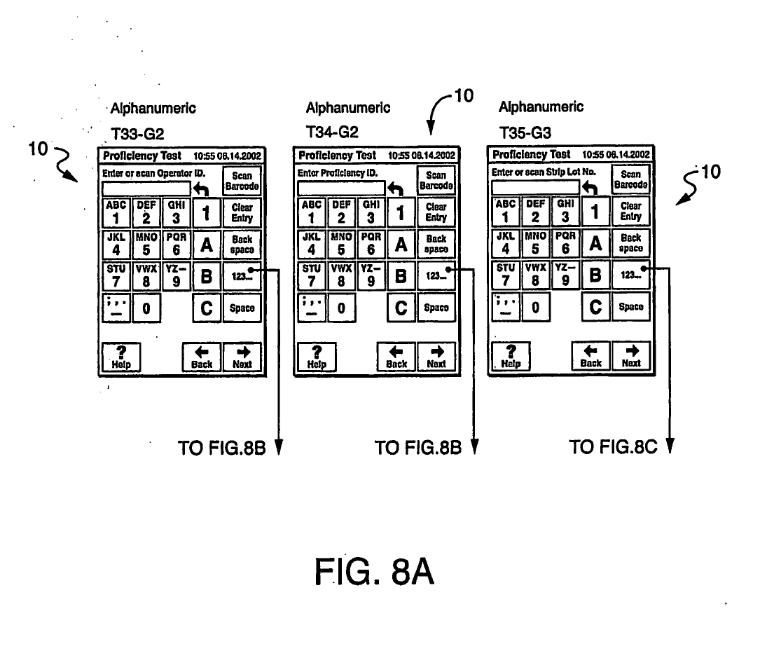

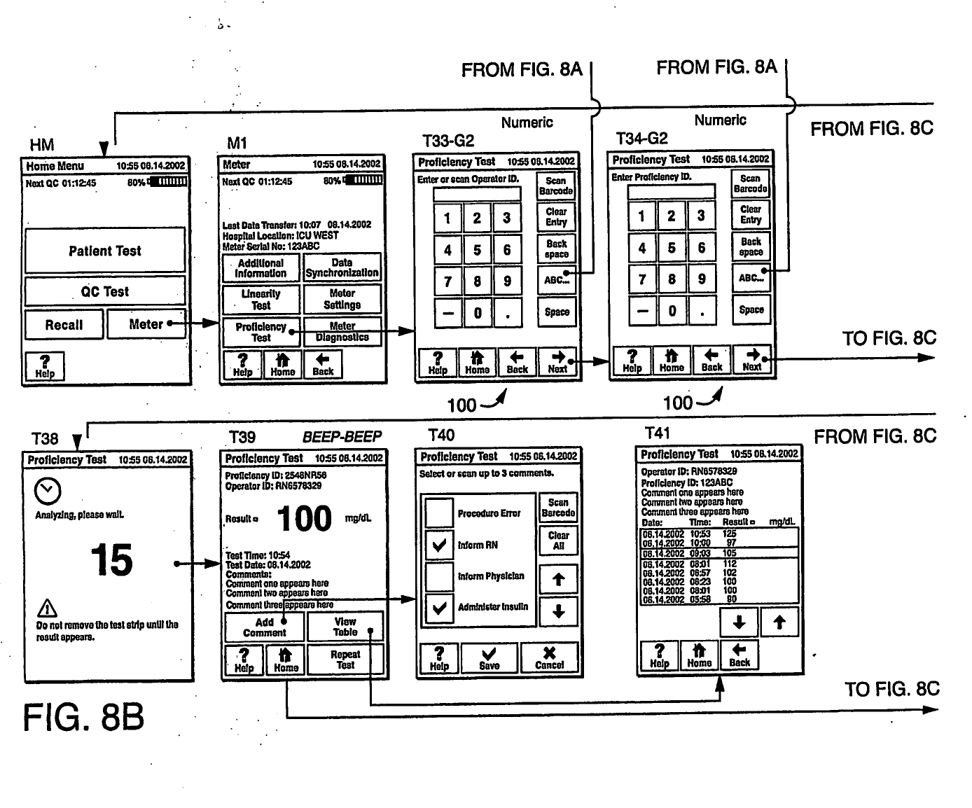

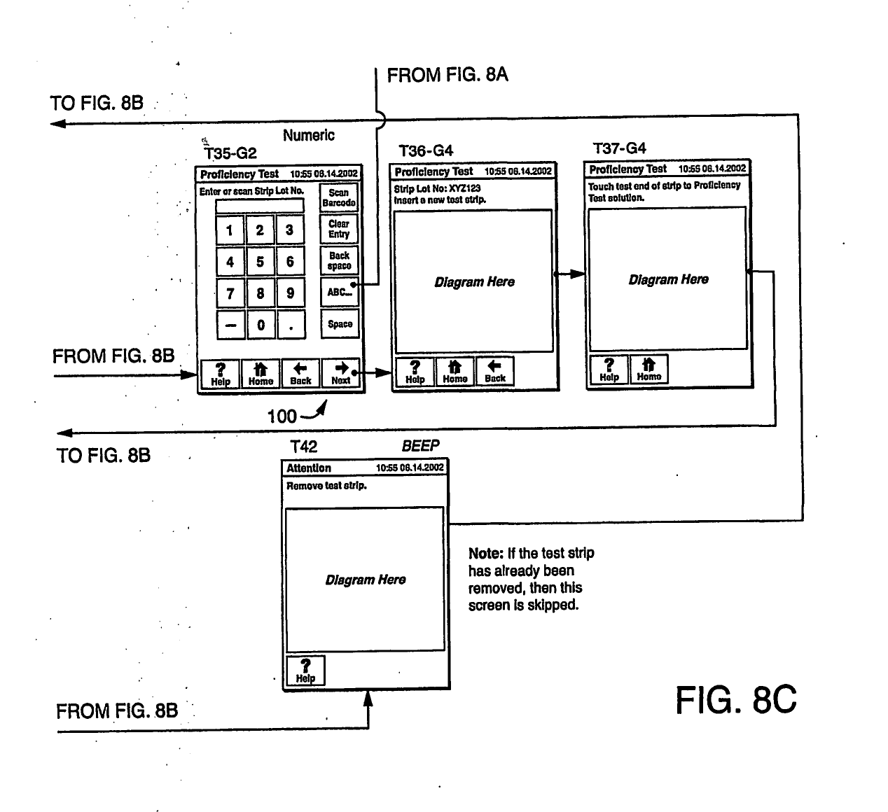

FIG. 8 is a series of screen shots showing an exemplary embodiment of a “proficiency test” GUI according to the present disclosure for display on the LCD 210 of the handheld glucose meter 200 of FIGS. 1-3 As shown in FIG. 8, the proficiency test GUIs are selected and run from the meter GUI, and the proficiency test offers a selection between the numeric GUI 100 and the alphanumeric GUI 10 of FIG. 4B when data entry is required from the user (alternatively, just the alphanumeric GUI 10 can be used if desired to receive alphabetical and numeric input).

Numerous further modifications and alternative embodiments of the disclosure will be apparent to those skilled in the art in view of the foregoing description. This description is to be construed as illustrative only, and is for the purpose of teaching those skilled in the art the best mode of carrying out the disclosure. The details of the apparatus and method may be varied substantially without departing from the spirit of the disclosure, and the exclusive use of all modifications which come within the scope of the appended claims is reserved.

What is claimed is:

1. A medical diagnostic apparatus comprising:
a display screen for displaying a graphical user interface (GUI) including virtual switches;
a user input device for allowing the virtual switches of the GUI to be selected;
a processor connected to the display screen and the user input device; and
a computer program having alphanumeric input instructions that cause the processor to display one of a numeric entry GUI and an alphanumeric entry GUI on the display screen, wherein selecting a predetermined virtual switch of the numeric GUI using the input device causes the alphanumeric GUI to be displayed, while selecting a predetermined virtual switch of the alphanumeric GUI causes the numeric GUI to be displayed.

2. An apparatus according to claim 1, wherein the numeric entry GUI includes ten virtual switches labeled with numeric characters 0-9 respectively.

3. An apparatus according to claim 1, wherein the alphanumeric entry GUI includes ten virtual switches labeled with numeric characters 0-9 respectively, and further includes a set of virtual switches including virtual switches labeled with alphabetical characters.

4. An apparatus according to claim 3, wherein the alphanumeric entry GUI includes multiple sets of virtual switches including virtual switches labeled with alphabetical characters, wherein each set is associated with one of the ten virtual switches labeled with numeric characters 0-9, and the computer program is programmed to display one of the sets when the numeric character virtual switch associated with the set is selected.

5. An apparatus according to claim 4, wherein each of the sets of virtual switches of the alphanumeric entry GUI includes a virtual switch labeled with the numeric character associated with the set.

6. An apparatus according to claim 5, wherein the sets of virtual switches of the alphanumeric entry GUI include:
a set of four virtual switches labeled with 1, A, B, C, respectively, associated with the virtual switch labeled with numeric character 1;
a set of four virtual switches labeled with 2, D, E, F, respectively, associated with the virtual switch labeled with numeric character 2;
a set of four virtual switches labeled with 3, G, H, I, respectively, associated with the virtual switch labeled with numeric character 3;
a set of four virtual switches labeled with 4, J, K, L, respectively, associated with the virtual switch labeled with numeric character 4;
a set of four virtual switches labeled with 5, M, N, 0, respectively, associated with the virtual switch labeled with numeric character 5;
a set of four virtual switches labeled with 6, P, Q, R, respectively, associated with the virtual switch labeled with numeric character 6;
a set of four virtual switches labeled with 7, S, T, U, respectively, associated with the virtual switch labeled with numeric character 7;
a set of four virtual switches labeled with 8, V, W, X, respectively, associated with the virtual switch labeled with numeric character 8; and
a set of three virtual switches labeled with 9, Y, Z, respectively, associated with the virtual switch labeled with numeric character 9.

7. An apparatus according to claim 1, wherein the predetermined virtual switch of the numeric GUI is labeled “ABC . . . ” and the predetermined virtual switch of the alphanumeric GUI is labeled “123 . . . ”.

8. An apparatus according to claim 1, wherein the user input device comprises a touch screen.

9. An apparatus according to claim 1, wherein the display screen comprises a liquid crystal display.

10. An apparatus according to claim 1, further comprising:
a port for receiving a fluid sample; and
a detector connected to the processor and adapted to measure a predetermined physical characteristic of the fluid sample received in the port;
wherein the processor is programmed to calculate a level of blood glucose in the fluid sample received in the port using the measurement of the physical characteristic of the fluid sample provide by the detector.

11. An apparatus according to claim 1, further comprising a bar code scanner connected to the processor.

12. A user interface for a medical diagnostic apparatus comprising:
a numeric entry graphical user interface (GUI) including a predetermined virtual switch and ten virtual switches labeled with numeric characters 0-9 respectively; and
an alphanumeric entry GUI including a predetermined virtual switch, ten virtual switches labeled with numeric characters 0-9 respectively, and a set of virtual switches including virtual switches labeled with alphabetical characters;
wherein selecting the predetermined virtual switch of the numeric GUI using an input device causes the alphanumeric GUI to be displayed, while selecting the predetermined virtual switch of the alphanumeric GUI causes the numeric GUI to be displayed.

13. A user interface according to claim 12, wherein the alphanumeric entry GUI includes multiple sets of virtual switches including the virtual switches labeled with alphabetical characters, wherein each set is associated with one of the ten virtual switches labeled with numeric characters 0-9, and the user interface displays one of the sets when the numeric character virtual switch associated with the set is selected.

14. A user interface according to claim 13, wherein each of the sets of virtual switches of the alphanumeric entry GUI includes a virtual switch labeled with the numeric character associated with the set.

15. A user interface according to claim 14, wherein the sets of virtual switches of the alphanumeric entry GUI include:
a set of four virtual switches labeled with 1, A, B, C, respectively, associated with the virtual switch labeled with numeric character 1;
a set of four virtual switches labeled with 2, D, E, F, respectively, associated with the virtual switch labeled with numeric character 2;
a set of four virtual switches labeled with 3, G, H, I, respectively, associated with the virtual switch labeled with numeric character 3;
a set of four virtual switches labeled with 4, J, K, L, respectively, associated with the virtual switch labeled with numeric character 4;
a set of four virtual switches labeled with 5, M, N, 0, respectively, associated with the virtual switch labeled with numeric character 5;
a set of four virtual switches labeled with 6, P, Q, R, respectively, associated with the virtual switch labeled with numeric character 6;
a set of four virtual switches labeled with 7, S, T, U, respectively, associated with the virtual switch labeled with numeric character 7;
a set of four virtual switches labeled with 8, V, W, X, respectively, associated with the virtual switch labeled with numeric character 8; and
a set of three virtual switches labeled with 9, Y, Z, respectively, associated with the virtual switch labeled with numeric character 9.

16. A user interface according to claim 12, wherein the predetermined virtual switch of the numeric GUI is labeled “ABC . . . ” and the predetermined virtual switch of the alphanumeric GUI is labeled “123 . . . ”.

17. A method for controlling a medical diagnostic apparatus comprising:
displaying graphical user interfaces (GUI) including virtual switches on a display screen of the medical diagnostic apparatus; and
allowing a user to select the virtual switches of the GUI through input devices of the medical diagnostic apparatus;
wherein the GUIs include a numeric entry GUI including a predetermined virtual switch and ten virtual switches labeled with numeric characters 0-9 respectively, and an alphanumeric entry GUI including a predetermined virtual switch, ten virtual switches labeled with numeric characters 0-9 respectively, and a set of virtual switches including virtual switches labeled with alphabetical characters, wherein the numeric GUI is displayed upon the predetermined virtual switch of the alphanumeric GUI being selected using the input device, and the alphanumeric GUI is displayed upon the predetermined virtual switch of the numeric GUI being selected using the input device.

18. A method according to claim 17, wherein the alphanumeric entry GUI is provided with multiple sets of virtual switches including virtual switches labeled with alphabetical characters, wherein each set is associated with one of the ten virtual switches labeled with numeric characters 0-9, and the computer program is programmed to display one of the sets when the numeric character virtual switch associated with the set is selected.

19. A method according to claim 18, wherein each of the sets of virtual switches of the alphanumeric entry GUI includes a virtual switch labeled with the numeric character associated with the set.

20. A method according to claim 19, wherein the sets of virtual switches of the alphanumeric entry GUI include:
a set of four virtual switches labeled with 1, A, B, C, respectively, associated with the virtual switch labeled with numeric character 1;
a set of four virtual switches labeled with 2, D, E, F, respectively, associated with the virtual switch labeled with numeric character 2;
a set of four virtual switches labeled with 3, G, H, I, respectively, associated with the virtual switch labeled with numeric character 3;
a set of four virtual switches labeled with 4, J, K, L, respectively, associated with the virtual switch labeled with numeric character 4;
a set of four virtual switches labeled with 5, M, N, 0, respectively, associated with the virtual switch labeled with numeric character 5;
a set of four virtual switches labeled with 6, P, Q, R, respectively, associated with the virtual switch labeled with numeric character 6;
a set of four virtual switches labeled with 7, S, T, U, respectively, associated with the virtual switch labeled with numeric character 7;
a set of four vitual switches labeled with 8, V, W, X, respectively, associated with the virtual switch labeled with numeric character 8; and
a set of three virtual switches labeled with 9, Y, Z, respectively, associated with the virtual switch labeled with numeric character 9.

21. A method according to claim 17, wherein the predetermined virtual switch of the numeric GUI is labeled “ABC . . . ” and the predetermined virtual switch of the alphanumeric GUI is labeled “123 . . . ”.

22. A medical diagnostic apparatus comprising:
a display screen for displaying a graphical user interface (GUI) including virtual switches;
a user input device for allowing the virtual switches of the GUI to be selected;
a processor connected to the display screen and the user input device; and
a computer program having alphanumeric input instructions that cause the processor to display an alphanumeric entry GUI on the display screen, wherein the alphanumeric GUI includes ten virtual switches labeled with numeric characters 0-9 respectively, and further includes multiple sets of virtual switches having virtual switches labeled with alphabetical characters, wherein each set is associated with one of the ten virtual switches labeled with numeric characters 0-9, and the computer program is programmed to display one of the sets when the numeric character virtual switch associated with the set is selected.

23. An apparatus according to claim 22, wherein each of the sets of virtual switches of the alphanumeric entry GUI includes a virtual switch labeled with the numeric character associated with the set.

24. An apparatus according to claim 2, wherein the sets of virtual switches of the alphanumeric entry GUI include:
a set of four virtual switches labeled with 1, A, B, C, respectively, associated with the virtual switch labeled with numeric character 1;
a set of four virtual switches labeled with 2, D, E, F, respectively, associated with the virtual switch labeled with numeric character 2;
a set of four virtual switches labeled with 3, G, H, I, respectively, associated with the virtual switch labeled with numeric character 3;
a set of four virtual switches labeled with 4, J, K, L, respectively, associated with the virtual switch labeled with numeric character 4;
a set of four virtual switches labeled with 5, M, N, 0, respectively, associated with the virtual switch labeled with numeric character 5;
a set of four virtual switches labeled with 6, P, Q, R, respectively, associated with the virtual switch labeled with numeric character 6;
a set of four virtual switches labeled with 7, S, T, U, respectively, associated with the virtual switch labeled with numeric character 7;
a set of four virtual switches labeled with 8, V, W, X, respectively, associated with the virtual switch labeled with numeric character 8; and
a set of three virtual switches labeled with 9, Y, Z, respectively, associated with the virtual switch labeled with numeric character 9.

25. An apparatus according to claim 22, wherein the user input device comprises a touch screen.

26. An apparatus according to claim 22, wherein the display screen comprises a liquid crystal display.

27. An apparatus according to claim 22, further comprising:
a port for receiving a fluid sample; and
a detector connected to the processor and adapted to measure a predetermined physical characteristic of the fluid sample received in the port;
wherein the processor is programmed to calculate a level of blood glucose in the fluid sample received in the port using the measurement of the physical characteristic of the fluid sample provide by the detector.

28. An apparatus according to claim 22, further comprising a bar code scanner connected to the processor.
